(19) United States
(12) Patent Application Publication
(10) Pub. No.: US 2012/0219984 A1
(43) Pub. Date: Aug. 30, 2012

(54) METHOD FOR THE ANAEROBIC TREATMENT OF A WASTEWATER AND ASSOCIATED DEVICE

(75) Inventors: Luis Castillo Rivera, Neuville Sur Oise (FR); Emmanuel Trouve, Montpellier (FR)

(73) Assignee: VEOLIA WATER SOLUTIONS & TECHNOLOGIES SUPPORT, Saint-Maurice (FR)

(21) Appl. No.: 13/508,142

(22) PCT Filed: Nov. 4, 2010
(86) PCT No.: PCT/FR10/52381

§ 371 Date: May 4, 2012

(30) Foreign Application Priority Data
Nov. 6, 2009 (FR) 0957887

(51) Int. Cl. C12Q 1/02; C12M 1/34

(52) U.S. Cl. 435/29; 435/287.1

(57) ABSTRACT

The invention relates to a device and a method for anaerobic treatment of wastewater in a biological reactor (100) involving at least one stabilised bacteria species including the following steps: measuring (150) a concentration, in an area linked to the reactor, of a reaction product inhibiting said at least one bacteria species; extracting (160) a fraction of the contents of the reactor when said concentration exceeds a predefined threshold; separating (300) said fraction into first and second sub-fractions, the first sub-fraction being depleted of said reaction product; and recirculating (310) the first sub-fraction in the reactor.

The invention relates to a method for the anaerobic treatment of a wastewater and associated device.

The invention falls within the field of systems for the biological treatment of urban and industrial wastewaters carrying a high concentration of organic material (more than 1 kg/m³, or even more than 3 kg/m³) and characterized by an elevated production of at least one by-product, for example volatile fatty acids (VFA). A threshold for characterizing such a production as an elevated VFA production is for example 200 mg per litre of treated water.

In the known devices, during the aerobic or anaerobic biological degradation of organic compounds contained in the effluents, there may be an accumulation of reactive organic products (or by-products) at concentrations capable of inhibiting the microorganisms, which can lead to a malfunction of the treatment system and to a drop in the production of methane in the case of anaerobic systems.

Two responses to the overloading of certain by-products are known. The first is stopping the feed of the raw influent or diluting it; and the second is the addition of chemical substances (for example calcium salts or soda) in order to precipitate the by-products.

These solutions are not always satisfactory on an industrial scale, as firstly, it is necessary to provide storage ponds and/or water for dilution and secondly, it is necessary to provide for regular flushing in order to avoid clogging problems.

Moreover, some of the reaction products are not recovered, although they could be put to use if extracted from the reaction mixture.

In this context, different methods, presented hereinafter, are known.

Document U.S. Pat. No. 3,711,392 discloses a method for the use of organic wastes involving a fermentation tank, a precipitation tank and an electrodialysis apparatus. The aim is the preparation of food products.

Document EP 1 236 688 discloses a biological treatment plant for wastewater, equipped with anaerobic digesters. A reactor has an inclined base allowing the removal of heavy metals and non-biodegradable products of the acidogenic bacteria by gravity.

Document U.S. Pat. No. 6,391,598 discloses a method for the preparation of fatty acid metal salts from an anaerobic digestive extract from a ruminant.

Document US 2005/0112735 (WO2005/035693) discloses a method for the production of biodiesel from wastewater treatment plant sludges, using in particular the techniques of filtration, centrifugation and gravity separation, as well as esterification of lipids.

The document by Kim et al., Desalination, 172, 2005, 119 gives results from experimental studies relating to the filtration performance of a system composed of a digester at 35° C. coupled with a ceramic microfiltration membrane—monolithic or tubular from 0.5 μm to 5 μm pore diameter—for extracting organic compounds dissolved in a sludge. The pH is adjusted with soda and hydrochloric acid.

These authors conclude that a pH comprised between 5 and 6 allows a better extraction of the organic components. A pore size of 1 μm is presented as optimum for the extraction of the dissolved organic components. The document by Camargo et al. (IX Latin American Symposium on

Anaerobic Digestion, October 2008) presents the results of experimental studies relating to the combination of an electrodialysis unit with an anaerobic reactor having a submerged micromembrane with a pore size of 10 μm.

These authors describe the operation of a process in the start-up phase, but not when stabilized. They did not give a technical solution with regard to the stabilization of the pH at around 7.

Moreover, the electrodialysis unit is used to adapt the microorganisms to the VFA in the reactor by controlled recycling of the VFA into the reactor, which can lead to an increase in the organic load. In this context, the technical problem faced is to reduce the presence of by-products having an inhibiting, or even preventive, effect on the formation of biogas in the reaction mixture without stopping the feed of organic material or introducing chemical reagents.

The aim is to improve the methane formation yield by extracting toxic (or inhibiting) products, in particular VFA, ammonia or H₂ that limit the formation of biogas.

To this end, a method is proposed for the stabilized anaerobic treatment of a wastewater in a biological reactor, involving at least one bacterial species, comprising the following steps:
measuring a concentration in an area linked to the reactor of a reaction product inhibiting said at least one bacterial species,
extracting a fraction from the contents of the reactor when said concentration exceeds a predefined threshold,
separating said fraction into a first and a second sub-fractions, the first sub-fraction being depleted of said reaction product, and
recycling the first sub-fraction into the reactor.

The different steps of the method can advantageously be regular, even semi-continuous or continuous.

The relative proportions of the flow rates of the first and second sub-fractions are chosen as a function of the concentration of reaction products (VFA or other inhibitor product) in the reaction mixture of the biological reactor and of the solubilization capacity of a dilution solution used in the separation process. In the case of one embodiment, 1 volume of dilution solution and 9 volumes of the reaction mixture are used.

Using the method according to the invention, an increased yield is obtained from the water treatment process, and the reactor can be dimensioned in a smaller size.

The biogas production, in particular methane, is a function of the converted organic load and therefore of the Total Organic Carbon (TOC) consumed. The yield of the process is measured by one or other of these two parameters (biogas production or TOC consumption).

According to an advantageous feature, said area linked to the reactor comprises an inner area of the reactor and a feed pipe of the wastewater to be treated, an outlet pipe and optionally a recycling pipe originating from the liquid/solid separation and/or originating from the effluent output.

Thus, the measurement points are, according to the embodiments, before the reactor, in the reactor, on a recycling pipe, or on an outlet pipe. The extraction of a fraction from the contents of the reactor is carried out either at the reactor itself, or at the outlet pipe, or at the recycling pipe, or at the feed pipe, or at the feed tank, before entry into the biological reactor, in particular if there is a significant production of the reaction product at this level.

This makes it possible to limit the arrival of inhibitor products into the feed reactor, before entry into the biological reactor, in particular if there is significant production at this level, and then into the biological reactor.

According to another advantageous feature, the reactor is fixed-bed, allowing it to contain the quantity of free biomass. It can also be a fluidized bed or free biomass reactor.

In alternative embodiments, the biological reactor contains, instead of the fixed bed, a packed bed, a suspended bed or a mixture of fixed biomass on inert supports and activated sludges.

According to an advantageous feature, between the extraction and the separation, a filtration step is carried out with a membrane having pores of a size less than 5 μm.

The presence of an intermediate step between the extraction and the electrodialysis or of an intermediate module between the extraction device and the electrodialysis device is optional.

It makes it possible for example to avoid clogging of the ion exchange membranes contained in an electrodialysis unit used for the separation, if the extracted liquid portion contains too much material in suspension.

In different variants, other types of liquid/solid separator are used, such as a hydrocylone, sand filter, settling tanks, screen, centrifuge.

According to an advantageous feature, said filtration step is carried out with a ceramic membrane having pores of diameter 1.2 μm.

This makes it possible to obtain satisfactory results in terms of operation of the process.

According to an advantageous feature, the reactor is a mesophilic or thermophilic reactor, said reaction product being a combination of volatile fatty acids, and said area being a liquid-phase area.

Thus, the temperature range of the reactor is from 25 to 40° C. or from 40 to 65° C., for the mesophilic and thermophilic microorganisms respectively.

The microorganisms present in an alternative embodiment are psychrophilic, and are active within a temperature range of 0 to 25° C.

The quantity and the type of organic material contained in the wastewater to be treated, the hydraulic retention time and the type of microorganism determine the level of production and accumulation of VFA.

According to an advantageous feature, the predefined threshold is at least 200 mg/L of VFA.

In an embodiment a drop in the production of CH₄ is noted, starting from 200 mg/L of VFA.

In an alternative embodiment, the extraction is initiated as a function of the energy costs of the start-up of the extraction process in relation to the energy gains resulting from the improvement in the method of production of CH₄.

If the start-up of the extraction process is more costly in energy than the gains in production of CH₄ obtained by the process, then the threshold value for initiation is revised upwards.

In an embodiment, a set value of 1 g/L is used. This value is an average value which is advantageous from the point of view of energy gain.

The measurement system interval is set by a clock which performs a measurement at a regular interval of the VFA concentration in an area of the reactor.

Thus, according to the algorithm which governs the operation of extracting the VFA outside the reactor, the extraction device is not started until the VFA concentration exceeds the threshold of 1 g/L.

In a particular embodiment scenario of the method of the invention, during its measurement operation performed at regular intervals, the sensor measures a value of 1.2 g/L, a value that is greater than 1 g/L, and on this basis initiates the start-up of the extraction process.

The VFA threshold value is furthermore set according to the degree of adaptation of the microorganisms to the VFAs and the cost/benefit ratio of the extraction process.

According to an advantageous feature, the separation is carried out using an electrodialysis unit.

According to an advantageous feature, the electrodialysis unit contains a CEM membrane (cation exchange membrane) and/or an AEM (anion exchange membrane).

In a preferred embodiment, an electrodialysis unit is used, constituted by cells comprising both an anionic membrane and a cationic membrane.

According to an advantageous feature, the pH of the reactor is kept between 6.5 and 7.5

This pH interval is optimum for allowing the development of the methane-generating microorganisms.

There is in addition an interaction between the electrodialysis unit and the pH of the solution introduced, since at the indicated pH values, the reaction products are found in their dissociated (electrically charged) form, making extraction by electrodialysis (application of an electric field) feasible.

According to an advantageous feature, the water is an effluent of the agri-food industry or an urban wastewater.

According to an advantageous feature, a monitored recycling pipe is used in order to proceed to doping the reactor with the second sub-fraction.

The invention therefore proposes a device for the stabilized anaerobic treatment of a wastewater in a biological reactor involving at least one bacterial species comprising means of:
measuring a concentration of an area linked to the reactor of a reaction product inhibiting said at least one bacterial species,
extracting a fraction from the contents of the reactor when said concentration exceeds a predefined threshold,
separating said fraction into a first and a second sub-fractions, the first sub-fraction being depleted of said reaction product, and
recycling the first sub-fraction into the reactor.

According to advantageous features, the device according to the invention is equipped with means or functions corresponding to the features previously mentioned with regard to the method according to the invention.

The invention will now be described in detail with reference to the attached figures.

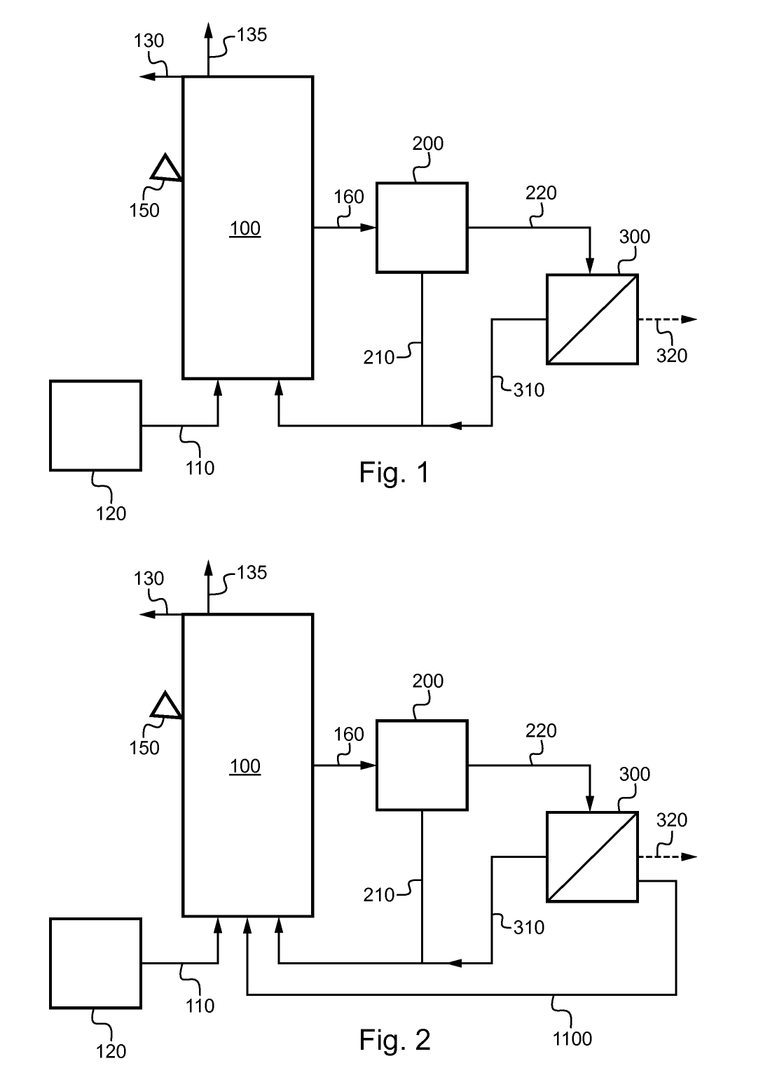

FIG. 1 shows an outline plan of the invention.

FIG. 2 shows a diagram of a particular embodiment of the invention.

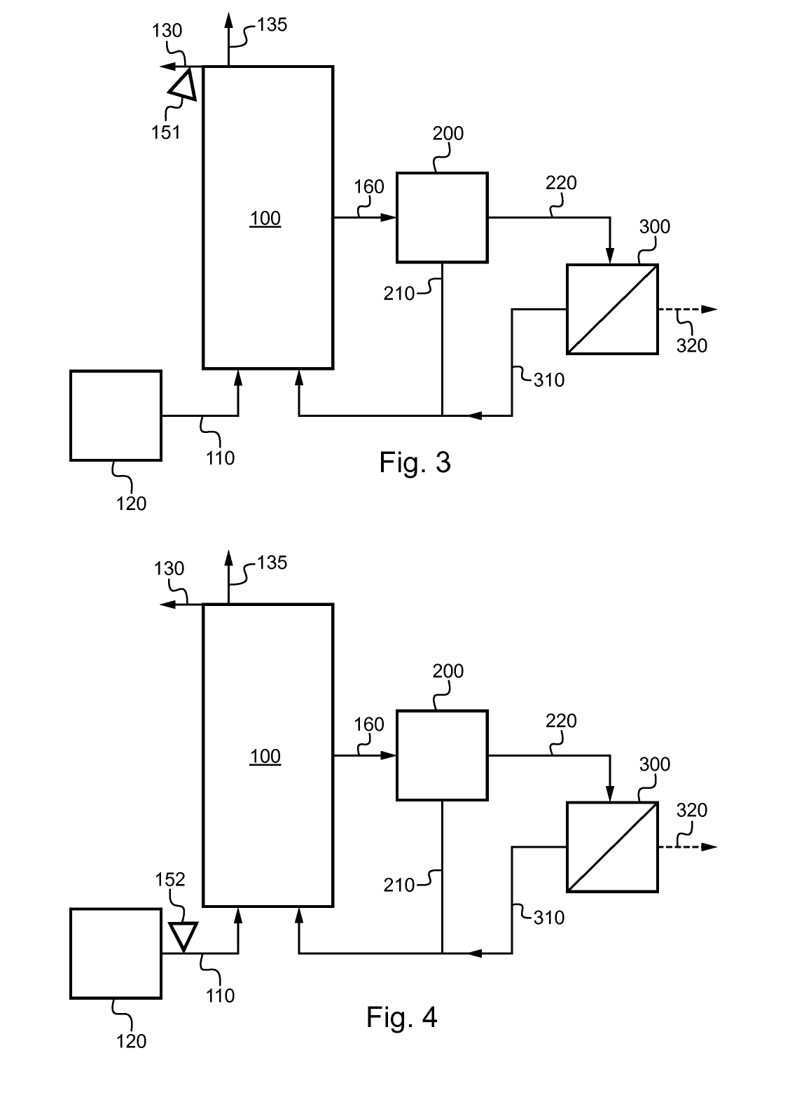

FIGS. 3 to 6 show further variants of the invention.

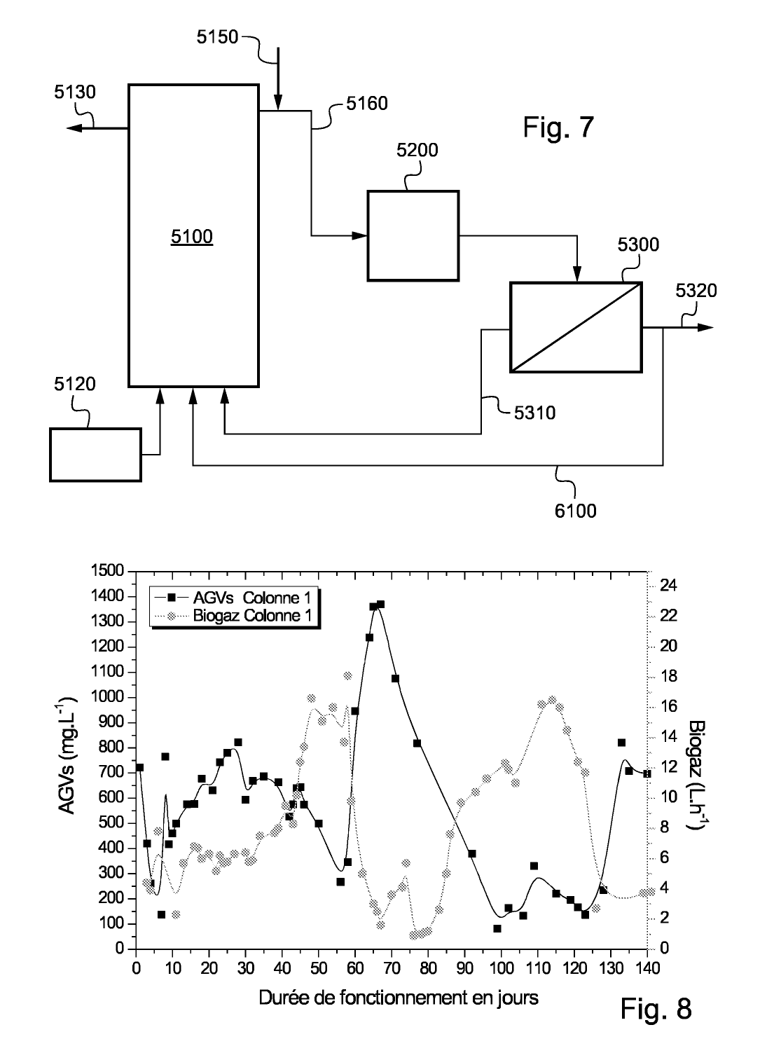

FIG. 7 shows a second embodiment of the invention.

FIGS. 8 to 10 show parameters measured during an implementation scenario of a method according to the invention.

With reference to FIG. 1, a wastewater treatment reactor 100 is fed from upstream, for example, by a feed tank (or flow equalization basin) 120, via a feed pipe 110. The wastewater is here a wastewater having an organic material concentration comprised between 20 and 30 kg/m³. With this order of magnitude of organic load, industrial wastewater must be involved. An urban wastewater, to which the invention can also be applied, generally contains an organic material concentration of around 1 kg/m³.

The reactor has an effluent outlet 130 and a methane outlet 135. It is a thermophilic biological reactor at 55° C., for example fixed-bed with plastic material as support, with ascending flow.

The reactor has an automatic device for regulating the pH which keeps the pH within a range of 6.5 to 7.5.

This device operates as follows: the probe of the pH-meter is placed in the reactor, then the pH-meter is linked to a computer containing a decision algorithm that by means of feed pumps, allows the control of the input of acid solution such as hydrochloric acid HCl, phosphoric acid H₂PO₄ or sulphuric acid H₂SO₄ or a basic solution, preferably soda NaOH, according to the pH in the reactor.

If the pH is greater than 7.5, then acid is injected until the pH returns to the required range, namely 6.5 to 7.5. If the pH is less than 6.5, then a basic solution is injected until the pH returns to within the range. If the pH is in the range 6.5-7.5, then the pumps do not operate. Thus the pH inside the reactor is kept close to a neutral value. It has been found that this makes it possible to promote the presence of dissociated VFAs, which appear to be easier to separate by electrochemical methods.

In a manner that is complementary to the measurement of the pH, the redox potential, measured for example using a redox meter, in the reactor must preferably be kept below a threshold value of −200 mV. Keeping the redox potential below said threshold value, well known to a person skilled in the art, allows an optimum development of the methane-producing bacteria.

A measurement of the concentration of volatile fatty acids (VFA) is carried out in the reactor at a measurement point 150, using an in-line analyzer. The latter monitors the position of the VFA concentration, measured in acetic acid equivalent, with respect to the threshold value of 1 g/L.

When the VFA concentration exceeds the indicated threshold, an extraction pipe 160 continuously extracts a fraction from the volume of the reactor 100 and conveys it to a solid/liquid separator 200 which here is a microfiltration system with ceramic membranes of 1.2 μm pore diameter (according to the variants, it is possible to perform a microfiltration or an ultrafiltration). It is noted that this separator is optional. Various means are used for carrying out the liquid/solid separation according to embodiments, such as a hydrocylone, centrifuge, sand filter, settling tanks, screen.

It is noted that the method for measurement and extraction is carried out using an algorithm, allowing the optimization of biogas production and the mineralization of the organic compounds.

The extraction flow rate, in an embodiment is less than or equal to 10 times the feed flow rate of the biological reactor.

A pipe 210 carries the solids separated by the separator to the reactor 100. A pipe 220 carries the liquid filtrate to an electrodialysis unit 300, which produces two sub-fractions, one of which is VFA-depleted, and the other enriched.

The proportions are functions of the VFA concentration (or other inhibitor product) in the reaction mixture of the biological reactor and of the solubilization capacity of the dilution solution used. In the case of the tests performed, 1 volume of dilution solution and 9 volumes of the reaction mixture were required.

The depleted fraction is conveyed to the reactor by the pipe 310 and the enriched fraction is removed from the system by the pipe 320, to undergo procedures that do not pertain to the present description. In an implementation scenario of the method according to the invention, the organic material concentration applied was comprised between 2 and 100 kg/m3.day. The hydraulic retention time was comprised between 18 and 96 hours. The hydraulic retention time is the average residence time of the effluent in the reactor.

FIG. 8 shows the impact of the accumulation of the VFAs on the biogas production (methane). It is noted that the greater the accumulation of VFA, the lower the biogas production. The production of biogas and the VFA concentration are shown on the y-axis (right axis and left axis respectively), the x-axis defining the duration of operation in days. This figure clearly shows the impact of the VFAs on the production of biogas. For VFA concentrations less than 400 mg.L⁻¹ and close to 200 mg.L⁻¹ the maximum biogas production is greater than 16 L.j⁻¹. However, when the VFA concentration is comprised between 400 and 800 mg.L⁻¹, the biogas production is reduced by more than 50% and more than 75% for VFA concentrations greater than 1000 mg.L⁻¹.

It is possible to reduce the VFA concentration of the effluent of the anaerobic reactor by more than 75% by using electrodialysis. The 1.2 μm filtration system was initiated when the VFA concentration reached a value in the reaction mixture of 1.2 g/L. The operating method implemented involves a constant voltage of 12.4 V and a variable amperage of I=1.2 A.

| | Duration of the test | VFA concentration |
|---|---|---|
| Operating method | (minutes) | (mg/L) in the effluent |
| Constant voltage at | 0 | 1200 |
| 12.4 V, variable | 2 | 636 |
| amperage | 5 | 510 |
| | 7 | 414 |
| | 9 | 270 |

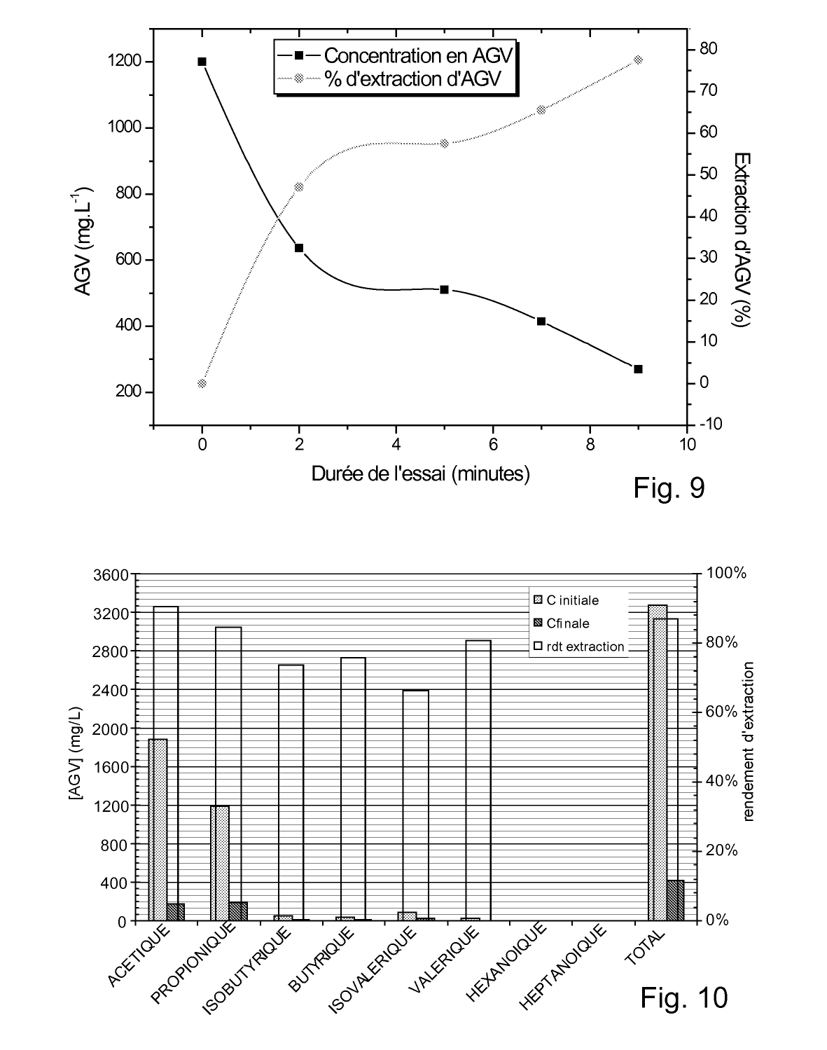

The VFA concentration is reduced by 50% after a time period of the order of 2 minutes, as shown in FIG. 9, which demonstrates the variations in the VFA concentration of the effluent as a function of time during the implementation scenario of the method according to the invention, with a voltage of 12.4 V. In this figure the VFA concentration is shown on the left-hand y-axis in mg/L, and the extraction of the VFAs as a percentage on the right-hand y-axis, the x-axis showing the duration of the test in minutes. The initial concentration is of the order of 1200 mg/L. It reduces in two phases, from 0 to 2 minutes to a value of 500 mg/L, then from 5 to 9 minutes, to a value of 250 mg/L.

Finally, FIG. 10 demonstrates that all the VFAs are extracted, which is shown by the lifting of the inhibition on the biogas production. In this figure, the initial concentration and the final concentration (left-hand y-axis, in mg/L), and the extraction yield (right-hand y-axis, as a percentage), are shown for acetic, propionic, isobutyric, butyric, isovaleric, valeric, hexanoic (absent) and heptanoic (absent) acids, as well as for the entire family of volatile fatty acids. The yields are always greater than 65%, and the total yield is close to 90%. The main acids concerned are acetic acid and propionic acid, their initial concentrations being of the order of 1200 to 1900 mg/L, with an associated extraction yield of the order of 85%.

With reference to FIG. 2, the pipe 1100 allows a controlled recycling of a concentrated solution of volatile fatty acids originating from the electrodialysis unit 300 (concentrated sub-fraction originating from the separation), thus causing doping, preferably at a low flow rate. Thus, steps are taken to use the extracted products for regulating the reactor load, making it possible to establish conditions favourable to the adaptation of the biomass over time to the desired exploitation compounds that inhibited the non-adapted biomass.

With reference to FIG. 3, an in-line analyzer placed at the measurement point 151 monitors the VFA concentration at the effluent outlet 130. The operation of the system is similar to that described with respect to FIG. 1, but the initiation threshold is adapted.

With reference to FIG. 4, an in-line analyzer placed at the measurement point 152 monitors the concentration of VFA or other by-products in the feed pipe 110, or in the feed tank 120, which is useful if the material to be treated contains inhibitor or toxic compounds. The operation of the system is similar to that described with respect to FIG. 1, but the initiation threshold is adapted.

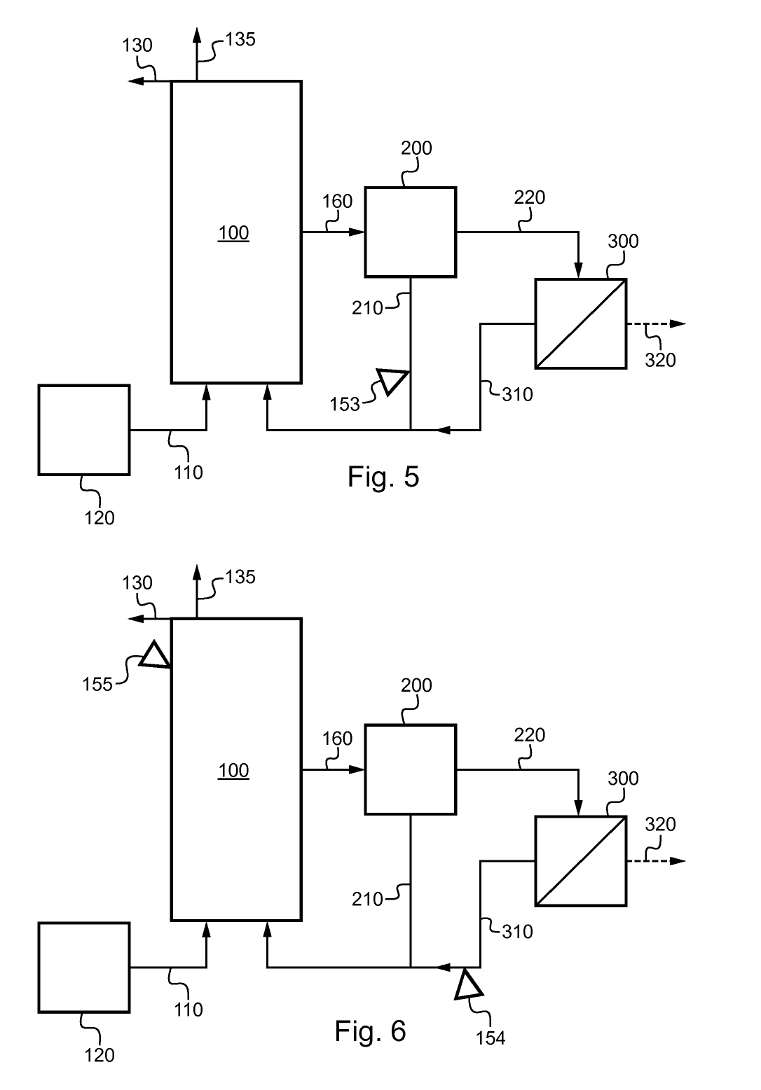

With reference to FIG. 5, an in-line analyzer placed at the measurement point 153 monitors the VFA concentration of the solids recycling pipe 210. The operation of the system is similar to that described with respect to FIG. 1, but the initiation threshold is adapted.

With reference to FIG. 6, an in-line analyzer placed at the measurement point 154 monitors the VFA concentration of the recycling pipeline of the depleted fraction 310.

The in-line analyzer 154 allows the VFA concentration of the VFA-depleted fraction to be verified and ensures that this concentration is below the initiation threshold of the electrodialysis unit.

In addition, an in-line analyzer 155 measures the VFA concentration of the reactor and controls the start-up of the electrodialysis unit 300.

The analyzer 155 makes it possible to regulate the operation of the pump (not shown), which conveys the liquid from the reactor 100 to the electrodialysis unit 300, as a function of the VFA concentration. The result of this in-line double measurement makes it possible to adjust the duration of operation of the electro-dialysis unit 300.

This configuration is particularly suitable in the case of effluents originating from the agri-food industry, in particular sugar refineries, which have a large quantity of easily-biodegradable carbon.

The operation of the system is similar to that described in relation to FIG. 1, but the initiation threshold is modified.

The biomass can be free (i.e. so-called flocculant sludges or so-called granular sludges) or fixed biomass (i.e. on supports of one type or another onto which the biomass is fixed). In the latter case, the supports on which the biomass is fixed can be contained between two grids or held by a grid acting as a ceiling or as a floor—according to the direction of the fluid, and in this case, the supports onto which the biomass is fixed, are arranged in a fixed bed. Alternatively, the supports onto which the biomass is fixed can be suspended, arranged in the form of a fluidized bed.

In different variants, the by-product extracted is ammonia (NH₃), ammonium ions (NH₄⁺) or dihydrogen (H₂), and the phase from which the extraction takes place is the liquid or gas phase. An example threshold for dihydrogen is 5.8 Pa, the extraction technique used thus being based on a membrane contactor (also valid for ammonia).

This variant constitutes an embodiment represented in FIG. 7, where a biological reactor 5100 is shown with an effluent feed 5120, an effluent outlet pipe 5130, a gaseous mixture extraction pipe 5160, an in-line measurement at the point 5150 of the methane, dihydrogen or ammonia concentration (in the vapour space above the liquid phase), for example, a pretreatment system 5200 (optional) and a by-products extraction reactor 5300, with an exploitables extraction pipe 5320, a recycling pipe 5310 for the fraction depleted in by-product and a monitored recycling pipe 6100 for concentrated by-product solution, the pipes 5310 and 6100 allowing recycling into the reactor.

The invention is not limited to the embodiments described but encompasses all variants available to a person skilled in the art.

1.-15. (canceled)

16. A method for the anaerobic treatment of a wastewater in a biological reactor, involving at least one bacterial species, comprising the following steps:
measuring a concentration in an area linked to the reactor of a reaction product inhibiting said at least one bacterial species
extracting a fraction from the contents of the reactor when said concentration exceeds a predefined threshold
separating said fraction into a first and a second sub-fractions, the first sub-fraction being depleted of said reaction product, and
recycling the first sub-fraction into the reactor.

17. The method according to claim 16, characterized in that said area linked to the reactor comprises an inner area of the reactor and a wastewater feed pipe, an outlet pipe or a recycling pipe.

18. The method according to claim 16, characterized in that the reactor is fixed-bed, fluidized-bed or free biomass.

19. The method according to claim 16, characterized in that between the extraction and the separation, a filtration step is carried out with a membrane having pores of a size less than 5 μm.

20. The method according to claim 19, characterized in that said filtration step is carried out with a ceramic membrane having pores of 1.2 μm diameter.

21. The method according to claim 16, characterized in that the reactor is a mesophilic or thermophilic reactor, said reaction product being a combination of volatile fatty acids, and said area being a liquid-phase area.

22. The method according to claim 16, characterized in that the predefined threshold is at least 200 mg/L.

23. The method according to claim 16, characterized in that the separation is carried out using an electrodialysis unit.

24. The method according to claim 23, characterized in that the electro-dialysis unit contains a cation exchange membrane and/or anion exchange membrane.

25. The method according to claim 16, characterized in that the pH of the reactor is kept between 6.5 and 7.5.

26. The method according to claim 16, characterized in that the water is an effluent of the agri-food industry or an urban wastewater.

27. The method according to claim 16, characterized in that a monitored recycling pipe is used to carry out a doping of the reactor with the second sub-fraction.

28. A device for the anaerobic treatment of a wastewater in a biological reactor involving at least one bacterial species comprising means for:
measuring a concentration in an area linked to the reactor of a reaction product inhibiting said at least one bacterial species
extracting a fraction from the contents of the reactor when said concentration exceeds a predefined threshold
separating said fraction into a first and a second sub-fractions, the first sub-fraction being depleted of said reaction product, and
recycling the first sub-fraction into the reactor.

29. A method for the anaerobic treatment of a wastewater in a biological reactor, involving at least one bacterial species, comprising the following steps:
measuring, using an in-line analyzer, a concentration in an area linked to the reactor of a reaction product inhibiting said at least one bacterial species
extracting a fraction from the contents of the reactor when said concentration exceeds a predefined threshold
separating said fraction into a first and a second sub-fractions, the first sub-fraction being depleted of said reaction product, and
recycling the first sub-fraction into the reactor.

30. A device for the anaerobic treatment of a wastewater in a biological reactor involving at least one bacterial species comprising means for:
measuring, using an in-line analyzer, a concentration in an area linked to the reactor of a reaction product inhibiting said at least one bacterial species
extracting a fraction from the contents of the reactor when said concentration exceeds a predefined threshold
separating said fraction into a first and a second sub-fractions, the first sub-fraction being depleted of said reaction product, and
recycling the first sub-fraction into the reactor.
